(19) United States
(12) Patent Application Publication
(10) Pub. No.: US 2002/0165168 A1
(43) Pub. Date: Nov. 7, 2002

(54) USE OF SUGAR DERIVATIVES AS ANTIMICROBIAL, ANTIMYCOTIC AND/OR ANTIVIRAL ACTIVE SUBSTANCES

(76) Inventors: JOACHIM BUNGER, HAUBACH (DE); GUNTHER SCHNEIDER, HAMBURG (DE); JORG SCHREIBER, HAMBURG (DE); STEPHAN TEICHMANN, WEDEL (DE); FLORIAN WOLF, HAMBURG (DE)

Correspondence Address: SPRUNG KRAMER SCHAEFER & BRISCOE, 660 WHITE PLAINS ROAD, TARRYTOWN, NY, 105915144

(21) Appl. No.: 09/091,602

(22) PCT Filed: Dec. 4, 1995
(86) PCT No.: PCT/EP96/05400

(30) Foreign Application Priority Data
Dec. 16, 1995 (DE) 195 47 160.1

(51) Int. Cl. A61K 31/70; A61K 31/715; A61K 31/47

(52) U.S. Cl. 514/23; 514/38; 514/53; 514/54

(57) ABSTRACT

Use of alkylated and/or acylated monosaccharides and/or oligosaccharides as antimicrobial, antimycotic and/or antiviral active ingredients.

The present invention relates to the use of substances, which are known per se, as substances which are active against bacteria, mycota and viruses. In particular embodiments, the present invention relates to cosmetic and dermatological formulations comprising such substances.

The healthy warm-blooded organism, in particular the healthy human skin, is populated with a large number of non-pathogenic microorganisms. This so-called microflora of the skin is not only harmless, it constitutes an important protection for defence against opportunistic or pathogenic microbes.

Bacteria belong to the prokaryotic single-cell organisms. They can be distinguished roughly according to their shape (sphere, cylinder, curved cylinder) and according to the structure of their cell wall (Gram-positive, Gram-negative). More precise classifications take additional account of the physiology of the organisms. Thus, aerobic, anaerobic and facultatively anaerobic bacteria exist. Some individuals are of medical importance in their properties as pathogenic microbes, and others in turn are completely harmless.

Substances which are active against bacteria have been known for a considerable length of time. For example, the term “antibiotics”, which cannot be applied to all substances having an antimicrobial action, can be dated back to the year 1941, although the first findings of penicillin were already observed in 1929. Antibiotics in the current sense are not suitable for all medical and certainly not all cosmetic applications, since the warm-blooded organism, that is to say, for example, the sick patient, is often also impaired in its metabolic functions during use in any manner.

One object of the present invention was thus to enrich the prior art in this direction, that is to say, in particular, to provide substances which are active against Gram-positive and/or Gram-negative bacteria without an unacceptable impairment to the health of the user being associated with the use of the substances.

Gram-negative microbes are, for example, Escherichia coli, Pseudomonas species and Enterobacteriaceae, such as, for example, Citrobacter freundii.

Gram-positive microbes also play a role in cosmetics and dermatology. In the case of impure skin, for example, bacterial secondary infections are of aetiological importance, in addition to other influences. One of the most important microorganisms connected with impure skin is Propionibacterium acnes.

Impure skin and/or comedones impair the well-being of those affected, even in mild cases. Since practically every adolescent is affected by impure skin to some degree, there is the need to remedy this state of affairs for many people.

A particular object of the present invention was thus to discover a substance or substance combination which is active against impure skin or Propionibacterium acnes.

In another embodiment, the present invention relates to cosmetic deodorants. Such formulations serve to eliminate body odour, which is formed when fresh perspiration, which is in itself odourless, is decomposed by microorganisms. Customary cosmetic deodorants are based on different active principles.

Both liquid deodorants, for example aerosol sprays, roll-ons and the like, and solid formulations, for example deodorant sticks, powders, powder sprays, intimate cleansing compositions and the like, are known and customary.

In so-called antiperspirants, the formation of perspiration can be suppressed by astringents—chiefly aluminium salts, such as aluminium hydroxychloride (aluminium chlorohydrate). Apart from denaturing the skin proteins, the substances used for this, however, have a drastic effect on the heat balance of the axillary region, depending on their dosage, and should at best be used in exceptional cases. The bacterial flora on the skin can be reduced by using antimicrobial substances in cosmetic deodorants. In the ideal case, only the odour-causing microorganisms should be effectively reduced. In practice, however, it has been found that the entire microflora of the skin may be impaired. The flow of perspiration itself is not influenced by this, and in the ideal case only microbial decomposition of the perspiration is temporarily stopped.

Combination of astringents with substances having an antimicrobial action in one and the same composition is also customary. However, the disadvantages of the two classes of active ingredient cannot be eliminated completely by this route.

Finally, body odour can also be masked by fragrances, a method which meets the aesthetic requirements of the consumer the least, since the mixture of body odour and perfume fragrance smells rather unpleasant.

Nevertheless, most cosmetic deodorants, and also most cosmetics overall, are perfumed, even if they comprise deodorizing active ingredients. Perfuming can also serve to increase consumer acceptance of a cosmetic product or to give a product a certain flair.

However, perfuming of cosmetic compositions comprising active ingredients, in particular cosmetic deodorants, is not infrequently problematic, because active ingredients and perfume constituents may occasionally react with one another and render each other inactive.

Deodorants should fulfil the following conditions:

1) They should have the effect of reliable deodorizing.

2) The natural biological processes of the skin should not be impaired by the deodorants.

3) The deodorants must be harmless in the event of an overdose or other use which is not as specified.

4) They should not become concentrated on the skin after repeated use.

5) They should be easy to incorporate into the customary cosmetic formulations.

Another object of the present invention was thus to develop cosmetic deodorants which do not have the disadvantages of the prior art. In particular, the deodorants should largely protect the microflora of the skin, but selectively reduce the number of microorganisms responsible for body odour.

It was furthermore an object of the invention to develop cosmetic deodorants which are distinguished by good skin tolerance. Under no circumstances should the deodorizing active principles become concentrated on the skin.

Another object was to develop cosmetic deodorants which harmonize with the largest possible number of customary cosmetic auxiliaries and additives, in particular with the perfume constituents which are important precisely in formulations having a deodorizing or antiperspirant action.

Yet another object of the invention was to provide cosmetic deodorants which are active over a relatively long period of time, and in particular of the order of at least half a day, without their action decreasing noticeably.

Finally, it was an object of the present invention to develop deodorizing cosmetic principles which can be incorporated as universally as possible into the most diverse presentation forms of cosmetic deodorants without being limited to one or a few specific presentation forms.

Fungi, also called mycota [μυκηζ=Greek for fungus] or mycobionts, in contrast to bacteria, belong to the eukaryotes. Eukaryotes are organisms of which the cells (eucytes), in contrast to those of the so-called prokaryotes (procytes), have a cell nucleus demarcated from the rest of the cytoplasm by a nuclear shell and nuclear membrane. The cell nucleus contains the genetic information stored in chromosomes.

Representatives of mycobionts include, for example, yeasts (Protoascomycetes), moulds (Plectomycetes), mildew (Pyrenomycetes), downy mildew (Phycomycetes) and toadstools (Basidiomycetes).

Fungi, including the Basidiomycetes, are not plant organisms, but like these have a cell wall, vacuoles filled with cell sap and a plasma flow which is easily visible under the microscope. They contain no photosynthetic pigments and are C-heterotrophic. They grow under aerobic conditions and obtain energy by oxidation of organic substances. Some representatives, for example yeasts, however, are facultative anaerobic organisms and are capable of producing energy by fermentation processes.

Dermatomycoses are diseases where certain types of fungi, in particular Dermatophytes, penetrate the skin and hair follicles. The symptoms of dermatomycoses are, for example, small blisters, exfoliation, rhagades and erosion, usually combined with itching or allergic eczema.

Dermatomycoses can essentially be divided into the following four groups: dermatophytoses (for example epidermophytosis, favus, microsporosis and trichophytosis), yeast mycoses (for example pityriasis and other mycoses caused by Pityrosporum, Candida infections, blastomycosis, Busse-Buschke disease, torulosis, Piedra alba, torulopsidosis and trichosporosis), mould mycoses (for example aspergillosis, cephalosporidosis, phycomycosis and scopulariopsidosis) and systemic mycoses (for example chromomycosis, coccidiomycosis and histoplasmosis).

The pathogenic and facultatively pathogenic microbes include, for example, from the group of yeasts, the Candida species (for example Candida albicans) and those of the family Pityrosporum. Pityrosporum species, in particular Pityrosporum ovale, are thought to be responsible for skin diseases such as pityriasis versi-color, seborrhoea in the form of seborrhoea oleosa and seborrhoea sicca, which manifest themselves above all as seborrhoea capitis (=dandruff), seborrhoeic eczema and pityrosporum folliculitis. Participation of Pityrosporum ovale in the development of psoriasis is a subject of discussion in the field.

All areas of the human skin can be affected by dermatomycoses. Dermatophytoses almost exclusively affect the skin, hair and nails. Yeast mycoses can also affect mucosa and internal organs, while systemic mycoses regularly extend to entire organ systems.

The regions of the body where moisture and heat can build up owing to clothing, jewellery or shoes are affected particularly often. Athlete's foot is thus one of the best-known and most widespread dermatomycoses. Fungal diseases of the finger-nail and toenail regions (onychomycoses), moreover, are particularly unpleasant.

Superinfections of the skin by fungi and bacteria are also not infrequent.

If a new infection with high microbe counts of one or more often physiological pathogens, for example Staphylococci, but often also non-physiological pathogens, for example Candida albicans, occurs with existing primary infection, i.e. the normal microbe population of the skin, and adverse influences coincide, “superinfection” of the affected skin may occur. The normal microflora of the skin (or of another organ of the body) becomes almost completely overgrown here by the secondary pathogen.

In cases which proceed favourably, such superinfections can manifest themselves in unpleasant skin symptoms (itching, unattractive external appearance), depending on the microbe in question. In cases which proceed adversely, however, they can lead to destruction of the skin over large areas, and in the worst case can even culminate in the death of the patient.

Superinfections of the type described above are secondary diseases which often occur, for example, with full-blown AIDS. Microbes, which are harmless per se—at any rate in low microbe densities—but are also decidedly pathogenic under certain circumstances, over-grow the healthy skin flora in this manner. Nevertheless, in AIDS cases other organs of the body are also affected by superinfections.

Such superinfections are also observed with a large number of dermatological diseases, for example atopic eczema, neurodermatitis, acne, seborrhoeic dermatitis or psoriasis. Many medical and therapeutic measures, for example radio- or chemotherapy of tumour diseases, immunosuppression induced by medicaments and caused as a side effect, or else systemic antibiotic treatment, as well as external chemical or physical influences (for example environmental pollution, smog, extreme exposure to UV light) also promote the occurrence of superinfections of the external and internal organs, in particular of the skin and of the mucosa.

Although it is easily possible to combat superinfections with antibiotics in an individual case, such substances usually have the disadvantage of unpleasant side effects. For example, patients are often allergic to penicillin, and for this reason a corresponding treatment would be out of the question in such a case.

Antibiotics administered topically furthermore have the disadvantage that they not only free the skin flora from secondary pathogens but also severely impair the skin flora, which is physiological per se, and the natural healing process is again slowed down in this way.

The object of the present invention was to eliminate the disadvantages of the prior art and to provide substances and formulations comprising such substances, by the use of which superinfections can be healed, the physiological skin flora suffering no significant losses.

In contrast to the prokaryotic and eukaryotic cellular organisms, viruses (virus=Latin for poison] are biological structures which require a host cell for biosynthesis. Extracellular viruses (also called “virions”) consist of a single- or double-stranded nucleic acid sequence (DNA or RNA) and a protein shell (called a capsid), which may be surrounded by an additional lipid-containing casing (envelope). The entire system of nucleic acid and capsid is also called a nucleocapsid. Viruses are classified conventionally according to clinical criteria, although now they are usually classified according to their structure, their morphology, and in particular according to the nucleic acid sequence.

Medically important genera of viruses are, for example, influenza viruses (Orthomyxoviridae family), lyssa-viruses (for example rabies, rhabdovirus family), enteroviruses (for example hepatitis A, Picornaviridae family) and hepadnaviruses (for example hepatitis B, Hepadnaviridae family).

Virucides, that is to say substances which kill viruses, do not exist in the true sense since viruses do not have their own metabolism. For this reason, there has also been debate as to whether viruses should be classified as organisms. Pharmacological intervention without damage to the unaffected cells is at any rate difficult. Possible action mechanisms in the fight against viruses are primarily interference in their replication, for example by blocking the enzymes present in the host cell which are important for replication. Furthermore, the release of the viral nucleic acids into the host cell can be prevented. In the context of the disclosure submitted here, terms such as “antiviral” or “active against viruses”, “virucidal” or similar are understood as meaning the property of a substance of protecting a single-cell or multicell organism from the harmful consequences of a viral infection, whether prophylactically or therapeutically, regardless of the actual action mechanism of the substance in the individual case.

However, the prior art lacks substances which are active against viruses and which furthermore cause no or no appreciable damage to the host organism.

An object of the present invention was thus to remedy this poor state of affairs, that is to say to discover substances which effectively protect a single- or multicell organism from the harmful consequences of a viral infection, whether prophylactically or therapeutically.

Surprisingly, it has been found, and therein lies the solution of these objects, that the use of alkylated and/or acylated monosaccharides and/or oligosaccharides has antimicrobial, antimycotic and/or antiviral active ingredients overcome the disadvantages of the prior art.

It has been found, surprisingly, that the active ingredients used according to the invention prevent the growth of Gram-positive and Gram-negative bacteria, mycobionts and viruses.

In particular, the active ingredients used according to the invention are capable of preventing the growth of yeasts, in particular of the Pityrosporum species, that is to say Pityrosporium ovale.

It has furthermore been found that the active ingredients used according to the invention prevent the development of seborrhoeic symptoms, in particular dandruff, and eliminate already existing seborrhoeic symptoms, in particular dandruff.

The active ingredients according to the invention furthermore are particularly suitable for use as a deodorizing active ingredient in cosmetic deodorants and against impure skin, mild forms of acne and Propioni-bacterium acnes.

Finally, it has been found that the active ingredients used according to the invention can prevent decay of organic matter, in particular cosmetic and dermatological formulations, due to attack by Gram-positive and Gram-negative bacteria, mycobionts and viruses, if they are added to these formulations.

The alkylated and/or acylated monosaccharides and/or oligosaccharides used according to the invention are sometimes also called alkyl or acyl monoglycosides or oligoglycosides, since the alkyl or acyl group is bonded glycosidally to the saccharide group.

The invention thus also relates to a method of combating mycobionts, characterized in that the active ingredients used according to the invention, if appropriate in a suitable cosmetic or dermatological carrier, are brought into contact with the region contaminated by mycobionts, and to a method for protecting organic products from attack by mycobionts, characterized in that the active ingredients used according to the invention are added in an active amount to these organic products.

The prior art consequently gave not the slightest indication of the use according to the invention as an antimycotic active principle.

It was furthermore surprising that the active ingredients used according to the invention have a particularly good action against the microbe Pityrosporum ovale, which is responsible for the development of dandruff, and related microbes. Formulations which are to be used against dandruff, for example antidandruff shampoos, are consequently a preferred embodiment of the present invention.

The alkylated and/or acylated monosaccharides and/or oligosaccharides used according to the invention are preferably covered by the generic structure Glyc-R, in which Glyc is a monosaccharide group, a disaccharide group or a trisaccharide group, and the radical R, which is a branched or unbranched saturated alkyl group or acyl group having 1-25 carbon atoms, which group is bonded glycosidally to the group Glyc.

The hexoses on which the alkyl and acyl monoglycosides used according to the invention are advantageously based are preferably chosen from the group consisting of aldohexoses, usually in their pyranoside form, thus allo(pyrano)ses, altro(pyrano)ses, gluco(pyrano)ses, manno (pyrano) ses, gulo (pyrano) ses, ido (pyrano) ses, galacto(pyrano)ses and talo(pyrano)ses, but the aldohexosyl derivatives present in furanoside form are also to be advantageously used, if necessary, according to the invention.

Parent (hexosyl)hexoses for disaccharides used according to the invention are advantageous and may preferably be chosen from the group consisting of pyranosylpyranoses and furanosylpyranoses having a 1,4-glycosidal or 1,6-glycosidal bond. They are preferably chosen from the group consisting of maltose, leucrose, lactose and sucrose.

Accordingly, the alkyl and acyl monoglycosides preferably used according to the invention can be characterized by the general structural formulae

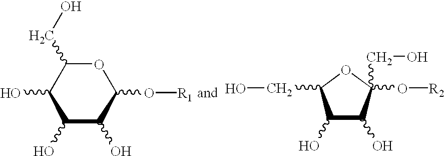

and the alkyl and acyl diglycosides and oligoglycosides used according to the invention are characterized by the general structural formulae

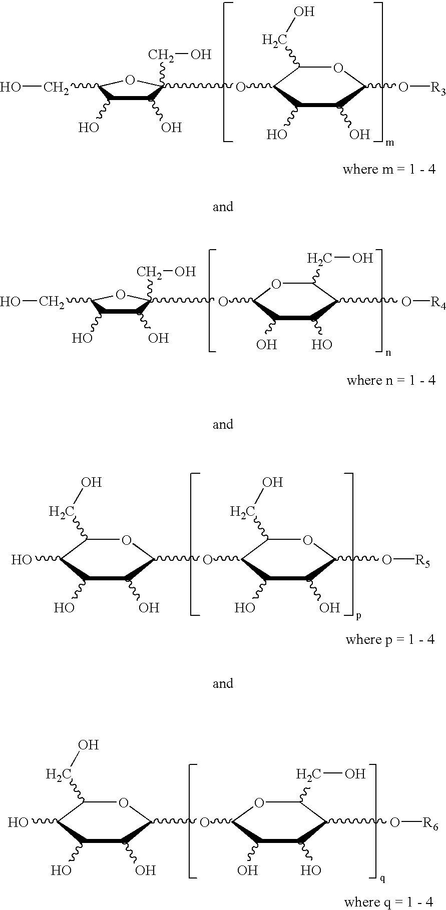

in which R₁-R₆ include branched or unbranched saturated alkyl groups or acyl groups having 1-25 carbon atoms.

The use of D-glycosides is advantageous, although L-glycosides or mixed D/L-glycosides can also be used advantageously for the purposes of the present invention.

Hexosylglycosides, on which D- or L-ketohexoses are based, thus psicose, fructose, sorbose or tagatose, usually present in their furanoside form, can also be advantageously used, if necessary, for the purposes of the present invention.

Alkyl and acyl glycosides which are used particularly advantageously according to the invention are chosen from the group consisting of β-D-octylglucopyranoside, β-D-nonylglucopyranoside, β-D-decylglucopyranoside, β-D-undecylglucopyranoside, β-D-dodecylglucopyranoside, β-D-tetradecylglucopyranoside and β-D-he-xadecylglucopyranoside.

Particular preference is given to octylglucopyranoside, β-D-nonylglucopyranoside and β-D-dodecylglucopyranoside, which are characterized in particular by very good action against Corynebacterium xerosis.

It is also advantageous to use natural or synthetic raw materials, auxiliaries and mixtures, which are characterized by an effective content of the active ingredients used according to the invention, for example Plantaren® 1200 (Henkel KGaA) and Oramix® NS 10 (Seppic).

It has been found, surprisingly, that the active ingredients used according to the invention prevent the growth of Gram-positive and Gram-negative bacteria, mycobionts and viruses.

In particular, the active ingredients used according to the invention are capable of preventing the growth of yeasts, in particular of the Pityrosporum species, that is to say Pityrosporium ovale.

It has furthermore been found that the active ingredients used according to the invention prevent the development of seborrhoeic symptoms, in particular dandruff, and eliminate already existing seborrhoeic symptoms, in particular dandruff.

The active ingredients used according to the invention furthermore are particularly suitable for use as a deodorizing active ingredient in cosmetic deodorants and against impure skin, mild forms of acne and Propioni-bacterium acnes.

Finally, it has been found that the active ingredients used according to the invention can prevent decay of organic matters, in particular cosmetic and dermatological formulations, due to attack by Gram-positive and Gram-negative bacteria, mycobionts and viruses, if they are added to these formulations.

The invention thus also relates to a method of combating mycobionts, characterized in that the active ingredients used according to the invention, if appropriate in a suitable cosmetic or dermatological carrier, are brought into contact with the region contaminated by mycobionts, and to a method for protecting organic products from attack by mycobionts, characterized in that the active ingredients used according to the invention are added in an active amount to these organic products.

The prior art consequently gave not the slightest indication of the use according to the invention as an antimycotic active principle.

It was furthermore surprising that the active ingredients used according to the invention have a particularly good action against the microbe Pityrosporum ovale, which is responsible for the development of dandruff, and related microbes. Formulations which are to be used against dandruff, for example antidandruff shampoos, are consequently a preferred embodiment of the present invention.

According to the invention, the active ingredients are preferably used in cosmetic or dermatological compositions in a content of 0.005-50.0% by weight, in particular 0.01-20.0% by weight, based on the total weight of the composition. The compositions advantageously comprise 0.02-10.0% by weight, particularly preferably 0.02-5.0% by weight, of the active ingredients used according to the invention, very particularly advantageously 0.5-3.0% by weight, in each case based on the total weight of the composition.

The active ingredients used according to the invention can be incorporated without difficulties into common cosmetic or dermatological formulations, advantageously into pump sprays, aerosol sprays, creams, ointments, tinctures, lotions, nail care products (e.g. nail varnishes, nail varnish removers, nail balsams) and the like.

It is also possible and in some instances advantageous to combine the active substances used according to the invention with other active substances, for example with other antimicrobial, antimycotic or antiviral substances.

It is advantageous to buffer the compositions according to the invention. A pH range from 3.5-7.5 is advantageous. It is particularly favourable to choose the pH within a range from 4.0-6.5.

The cosmetic and/or dermatological formulations according to the invention can have the customary composition and can be used for treating the skin and/or the hair in the sense of a dermatological treatment or a treatment in the sense of care cosmetics. They can however also be used in make-up products in decorative cosmetics.

For use, the cosmetic and dermatological formulations according to the invention are applied to the skin and/or the hair in an adequate amount in the manner customary for cosmetics and dermatological products.

Those cosmetic and dermatological formulations which are in the form of a sunscreen are advantageous. These advantageously additionally comprise at least one UVA filter and/or at least one UVB filter and/or at least one inorganic pigment.

Cosmetic formulations according to the invention for the protection of the skin against UV rays can be in various forms, such as are usually used for this type of formulation. For example, they can be a solution, an emulsion of the water-in-oil (W/O) type or of the oil-in-water (O/W) type, or a multiple emulsion, for example of the water-in-oil-in-water (W/O/W) type, a gel, a hydrodispersion, a solid stick, or also an aerosol.

The cosmetic formulations according to the invention can comprise cosmetic auxiliaries such as are usually used in such formulations, e.g. preservatives, bactericides, antioxidants, perfumes, antifoams, colorants, pigments which have a colouring effect, thickeners, surfactants, emulsifiers, emollients, moisturizers and/or humectants, fats, oils, waxes or other customary constituents of a cosmetic formulation, such as alcohols, polyols, polymers, foam stabilizers, electrolytes, organic solvents or silicone derivatives.

If the cosmetic or dermatological formulation is a solution or lotion, solvents which may be used are:

water or aqueous solutions;

oils, such as triglycerides of capric or of caprylic acid, but preferably castor oil;

fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;

alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the abovementioned solvents are used. In the case of alcoholic solvents, water can be a further constituent.

According to the invention, favourable antioxidants which can be used are all the antioxidants which are suitable or customary for cosmetic and/or dermatological applications.

The antioxidants are advantageously chosen from the group consisting of amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotenoids, carotenes (for example α-carotene, β-carotene, lycopene) and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulphoximine compounds (for example buthionine-sulphoximines, homocysteine-sulphoximine, buthionine sulphones, penta-, hexa- and heptathionine-sulphoximine) in very low tolerated doses (for example pmol to pmol/kg), and furthermore (metal) chelating agents (for example α-hydroxy-fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutic acid and derivatives thereof, ferulic acid and derivatives thereof, butylated hydroxytoluene, butylated hydroxyanisole, nordihydroguaiac resin acid, nordihydroguaiaretic acid, trihydroxybutyrophenone, uric acid and derivatives thereof, mannose and derivatives thereof, zinc and derivatives thereof (for example ZnO, ZnSO₄), selenium and derivatives thereof (for example selenium methionine), stilbenes and derivatives thereof (for example stilbene oxide, trans-stilbene oxide) and the derivatives of these active ingredients mentioned which are suitable according to the invention (salts, esters, ethers, sugars, nucleotides, nucleosides, peptides and lipids).

The amount of the antioxidants (one or more compounds) in the formulations is preferably from 0.001 to 30% by weight, particularly preferably 0.05-20% by weight, in particular 1-10% by weight, based on the total weight of the formulation.

If vitamin E and/or derivatives thereof is or are the antioxidant or antioxidants, it is advantageous to choose the particular concentrations thereof from the range 0.001-10% by weight, based on the total weight of the formulation.

If vitamin A or vitamin A derivatives or carotenes or derivatives thereof is or are the antioxidant or antioxidants, it is advantageous to choose their particular concentrations from the range 0.001-10% by weight, based on the total weight of the formulation.

Emulsions according to the invention are advantageous and comprise, for example, the specified fats, oils, waxes and other fatty substances, and water and an emulsifier, such as is customarily used for such a type of formulation.

Gels according to the invention usually comprise alcohols of low C number, e.g. ethanol, isopropanol, 1,2-propanediol, glycerol and water and an abovementioned oil in the presence of a thickener which in the case of oily-alcoholic gels is preferably silicon dioxide or an aluminium silicate, and in the case of aqueous-alcoholic or alcoholic gels is preferably a polyacrylate.

Solid sticks according to the invention comprise, for example, natural or synthetic waxes, fatty alcohols or fatty acid esters. Preference is given to lip care sticks and deodorizing sticks (“Deo-Sticks”).

Suitable propellants for cosmetic or dermatological formulations according to the invention which can be sprayed from aerosol containers are the usual known, readily volatile, liquefied propellants, for example hydrocarbons (propane, butane, isobutane), which can be used on their own or in mixtures with one another. Compressed air is also advantageous.

The person skilled in the art obviously knows that there are propellent gases which are non-toxic per se and which would in principle be suitable for the present invention, but which, because of their harmful effect on the environment or other accompanying circumstances, should be avoided, in particular fluorocarbons and chlorofluorocarbons (CFCs).

The formulations according to the invention can preferably also comprise substances which absorb UV radiation in the UVB region, the total amount of filter substances being, for example, from 0.1% by weight to 30% by weight, preferably from 0.5 to 10% by weight, in particular from 1 to 6% by weight, based on the total weight of the formulation, in order to provide cosmetic formulations which protect the skin from the entire region of ultraviolet radiation. They can also be used as sunscreen.

The UVB filters can be oil-soluble or water-soluble. Examples of oil-soluble substances which can be mentioned are:

3-benzylidenecamphor and its derivatives, preferably 3-(4-methylbenzylidene)camphor;

4-aminobenzoic acid derivatives, preferably 2-ethylhexyl 4-(dimethylamino)benzoate, amyl 4-(dimethylamino)benzoate;

esters of cinnamic acid, preferably 2-ethylhexyl 4-methoxycinnamate, isopentyl 4-methoxycinnamate;

esters of salicylic acid, preferably 2-ethylhexyl salicylate, 4-isopropylbenzyl salicylate, homomenthyl salicylate;

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably di(2-ethylhexyl) 4-methoxybenzalmalonate;

2,4,6-trianilino-(p-carbo-2′-ethyl-1′-hexyloxy)-1,3,5-triazine.

Water-soluble substances are advantageously:

2-phenylbenzimidazole-5-sulphonic acid and its salts, for example sodium, potassium or triethanolammonium salts;

sulphonic acid derivatives of benzophenones, preferably 2-hydroxy-4-methoxybenzophenone-5-sulphonic acid and its salts;

sulphonic acid derivatives of 3-benzylidene camphor, such as for example 4-(2-oxo-3-bornylidenemethyl)benzenesulphonic acid, 2-methyl-5-(2-oxo-3-bornylidenemethyl)sulphonic acid and its salts.

The list of given UVB filters which can be used according to the invention is of course not intended to be limiting.

It can also be advantageous to use UVA filters which are usually present in cosmetic and/or dermatological formulations in the formulations according to the invention. Such substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. Formulations which contain these combinations are also a subject-matter of the invention. The same amount of UVA filter substances which were given for UVB filter substances can be used.

Cosmetic and/or dermatological formulations according to the present invention can also contain inorganic pigments which are usually used in the cosmetics industry for the protection of skin against UV radiation. These are oxides of titanium, zinc, iron, zirconium, silicon, manganese, aluminium, cerium and mixtures thereof, and modifications in which the oxides are the active agents. Pigments based on titanium dioxide are particularly preferred. The quantities given for the above combinations can be used.

Cosmetic formulations for hair care are, for example, shampoo compositions, formulations which are used when rinsing the hair before or after shampooing, before or after permanent wave treatment or before or after colouring or bleaching the hair, formulations for blow-drying or setting the hair, formulations for colouring or bleaching, a styling and treatment lotion, a hair lacquer or a permanent wave composition.

The cosmetic formulations comprise active ingredients and auxiliaries as are usually used for this type of formulation for hair care and hair treatment.

The auxiliaries used are preservatives, surfactants, antifoams, emulsifiers, thickeners, fats, oils, waxes, organic solvents, bactericides, perfumes, colorants or pigments, the task of which is to colour the hair or the formulation itself, electrolytes and formulations to prevent the hair becoming greasy.

Cosmetic formulations which are a shampoo composition or a wash, shower or bath formulation preferably comprise at least one anionic, nonionic or amphoteric surfactant or mixtures thereof, active ingredients according to the invention and auxiliaries as are usually used for this purpose.

Examples of surfactants which can be used advantageously according to the invention are conventional soaps, for example fatty acid salts of sodium, alkyl sulphates, alkyl ether sulphates, alkane- and alkylbenzenesulphonates, sulphoacetates, sulphobetaines, sarcosinates, amidosulphobetaines, sulphosuccinates, sulphosuccinic acid monoesters, alkyl ether carboxylates, protein-fatty acid condensates, alkylbetaines and amidobetaines, fatty acid alkanolamides and polyglycol ether derivatives.

The surfactant can be present in a concentration between 1% by weight and 50% by weight in the shampoo composition or the wash, shower or bath preparation.

If the cosmetic or dermatological formulation is in the form of a lotion which is rinsed out and used, for example, before or after colouring, before or after shampooing, between two shampooing steps, or before or after a permanent wave treatment, it comprises, for example, aqueous or aqueous-alcoholic solutions, which, if desired, comprise surfactants, preferably nonionic or cationic surfactants, the concentration of which may lie between 0.1 and 10% by weight, preferably between 0.2 and 5% by weight. This cosmetic or dermatological preparation may also be an aerosol comprising the customary auxiliaries used for this purpose.

A cosmetic formulation in the form of a lotion which is not rinsed out, in particular a lotion for setting the hair, a lotion which is used when blow-drying the hair, a styling and treatment lotion, is generally an aqueous, alcoholic or aqueous-alcoholic solution and comprises at least one cationic, anionic, nonionic or amphoteric polymer or mixtures thereof, and active ingredients according to the invention. The amount of active ingredients according to the invention used is, for example, between 0.1 and 10% by weight, preferably between 0.1 and 3% by weight.

Cosmetic and dermatological formulations for the treatment and care of hair which comprise the active ingredients used according to the invention may be in the form of emulsions of the nonionic or anionic type. As well as comprising water, nonionic emulsions comprise oils or fatty alcohols, which may, for example be polyethoxylated or polypropoxylated, or mixtures of the two organic components. These emulsions comprise, if desired, cationic surfactants.

Cosmetic and dermatological formulations for the treatment and care of the hair can be in the form of gels, which, in addition to active ingredients used according to the invention and solvents customarily used for this purpose, also comprise organic thickeners, for example gum arabic, xanthan gum, sodium alginate, cellulose derivatives, preferably methylcellulose, hydroxymethylcellulose, hydroxyethylcellulose, hydroxypropylcellulose or hydroxypropylmethylcellulose, or inorganic thickeners, e.g. aluminium silicates, such as, for example, bentonites, or a mixture of polyethylene glycol and polyethylene glycol stearate or distearate. The thickener is present in the gel, for example in an amount between 0.1 and 30% by weight, preferably between 0.5 and 15% by weight.

The amount of the active ingredients used according to the invention in a product intended for the hair is preferably from 0.01% by weight to 10% by weight, in particular from 0.5% by weight to 5% by weight, based on the total weight of the formulations.

The examples below serve to illustrate the present invention without limiting it.

### EXAMPLE 1

| W/O cream | I | II |
|---|---|---|
| Paraffin oil | 10.00 | 10.00 |
| Ozokerite | 4.00 | 4.00 |
| Vaseline | 4.00 | 4.00 |
| Vegetable oil | 10.00 | 10.00 |
| Wool wax alcohol | 2.00 | 2.00 |
| Aluminium stearate | 0.40 | 0.40 |
| Octylglucoside | 3.00 | — |
| Sucrose laurate | — | 3.00 |

| | |
|---|---|
| Perfume, preservatives | q.s. |
| Water, deionized | to 100.00 |
| pH: | to 5.5-6.0. |

### EXAMPLE 2

| O/W lotion | I | II |
|---|---|---|
| Paraffin oil | 5.00 | 5.00 |
| Isopropyl palmitate | 5.00 | 5.00 |
| Cetyl alcohol | 2.00 | 2.00 |
| Beeswax | 2.00 | 2.00 |
| Ceteareth-20 | 2.00 | 2.00 |
| PEG-20-glyceryl stearate | 1.50 | 1.50 |
| Glycerol | 3.00 | 3.00 |
| Plantaren ® 1200 | 5.00 | — |
| Decylglucoside | — | 5.00 |

| | |
|---|---|
| Perfume, preservatives | q.s. |
| Water, deionized | to 100.00 |
| pH: | to 5.5-6.0 |

### EXAMPLE 3

| Skin oil | I | II |
|---|---|---|
| Cetyl palmitate | 3.00 | 3.00 |
| C₁₂₋₁₅-alkyl benzoate | 2.00 | 2.00 |
| Polyisobutene | 10.00 | 10.00 |
| Squalane | 2.00 | 2.00 |
| Plantaren ® 2000 | 5.00 | — |
| Oramix ® NS 10 | — | 5.00 |

| | |
|---|---|
| Perfume, preservatives | q.s. |
| Paraffin oil | to 100.00 |

### EXAMPLE 4

| Lipstick | I | II |
|---|---|---|
| Ceresine | 8.00 | 8.00 |
| Beeswax | 4.00 | 4.00 |
| Carnauba wax | 2.00 | 2.00 |
| Vaseline | 40.00 | 40.00 |
| Hydrogenated castor oil | 4.00 | 4.00 |
| Caprylic/capric triglyceride | 6.00 | 6.00 |
| Plantaren ® 1200 | 2.00 | — |
| Sucrose myristate | — | 2.00 |

| | |
|---|---|
| Perfume, preservatives | q.s. |
| Paraffin oil | to 100.00 |

### EXAMPLE 5

| Care mask | I | II |
|---|---|---|
| PEG-50 lanolin | 0.50 | 0.50 |
| Glyceryl stearate | 2.00 | 2.00 |
| Sunflower kernel oil | 3.00 | 3.00 |
| Bentonite | 8.00 | 8.00 |
| Kaolin | 35.00 | 35.00 |
| Zinc oxide | 5.00 | 5.00 |
| Glucose caprylate | 2.00 | — |
| Oramix ® NS 10 | — | 2.00 |

| | |
|---|---|
| Perfume, preservatives | q.s. |
| Water, deionized | to 100.00 |
| pH: | ad 5.5-6.0 |

### EXAMPLE 6

| Syndet soap | I | II |
|---|---|---|
| Sodium lauryl sulphate | 30.00 | 30.00 |
| Sodium sulphosuccinate | 10.00 | 10.00 |
| Potassium cocoyl | | |
| hydrolysed collagen | 2.00 | 2.00 |
| Dimethicone copolyol | 2.00 | 2.00 |
| Paraffin | 2.00 | 2.00 |
| Maize starch | 10.00 | 10.00 |
| Talc | 10.00 | 10.00 |
| Glycerol | 3.00 | 3.00 |
| Plantaren ® 1200 | 3.00 | — |
| Oramix ® NS 10 | — | 3.00 |

| | |
|---|---|
| Perfume, preservatives | q.s. |
| Water, deionized | to 100.00 |
| pH: | to 5.5-6.0 |

### EXAMPLE 7

| Care shampoo | I | II |
|---|---|---|
| Sodium lauryl sulphate | 34.00 | 34.00 |
| Disodium lauryl | | |
| sulphosuccinate | 6.00 | 6.00 |
| Cocoamidopropylbetaine | 10.00 | 10.00 |
| Glycol distearate | 5.00 | 5.00 |
| Decylfructoside | 2.50 | — |
| Hexadecylglucoside | — | 2.50 |

| | |
|---|---|
| Perfume, preservatives | q.s. |
| Water, deionized | to 100.00 |
| pH: | to 5.5-6.0 |

### EXAMPLE 8

| Shaving foam | I | II |
|---|---|---|
| Stearic acid | 7.00 | 7.00 |
| Sodium lauryl sulphate | 3.00 | 3.00 |
| Stearyl alcohol | 1.00 | 3.00 |
| Glycerol | 5.00 | 5.00 |
| Triethanolamine | 3.60 | 3.60 |
| Sucrose caprinate | 1.50 | — |
| Sucrose myristate | — | 1.50 |

| | |
|---|---|
| Perfume, preservatives | q.s. |
| Water, deionized | to 100.00 |
| pH: | to 5.5-6.0 |

### EXAMPLE 9

| Aerosol spray | I | II |
|---|---|---|
| Octyldodecanol | 0.50 | 0.50 |
| Plantaren ® 1200 | 2.00 | — |
| Sucrose myristate | — | 2.00 |

| | |
|---|---|
| Perfume, preservatives | q.s |
| Ethanol | to 100.00 |

The liquid phase obtained by mixing together the particular constituents is transferred to an aerosol container together with a propane/butane mixture (2:7) in the ratio 39:61.

### EXAMPLE 10

| Roll-on gel | I | II |
|---|---|---|
| 1,3-Butylene glycol | 2.00 | 2.00 |
| PEG-40-hydrogenated | | |
| castor oil | 2.00 | 2.00 |
| Hydroxyethylcellulose | 0.50 | 0.50 |
| Plantaren ® 1200 | 5.00 | — |
| Decylglucoside | — | 5.00 |

| | |
|---|---|
| Perfume, preservatives | q.s. |
| Water, deionized | to 100.00 |
| pH: | to 5.5-6.0 |

1. Use of alkylated and/or acylated monosaccharides and/or oligosaccharides as antimicrobial, antimycotic and/or antiviral active ingredients.

2. Use according to claim 1, characterized in that the alkylated and/or acylated monosaccharide(s) is/are chosen from substances which are given by the general structural formulae 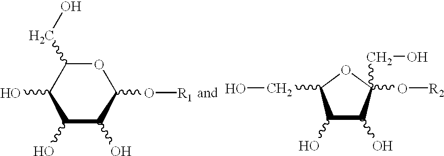
in which R₁ and/or R₂ include branched or unbranched saturated alkyl groups or acyl groups having 1-25 carbon atoms.

3. Use according to claim 1, characterized in that the alkylated and/or acylated disaccharide(s) or oligo-glucosides are chosen from substances which are given by the general structural formulae 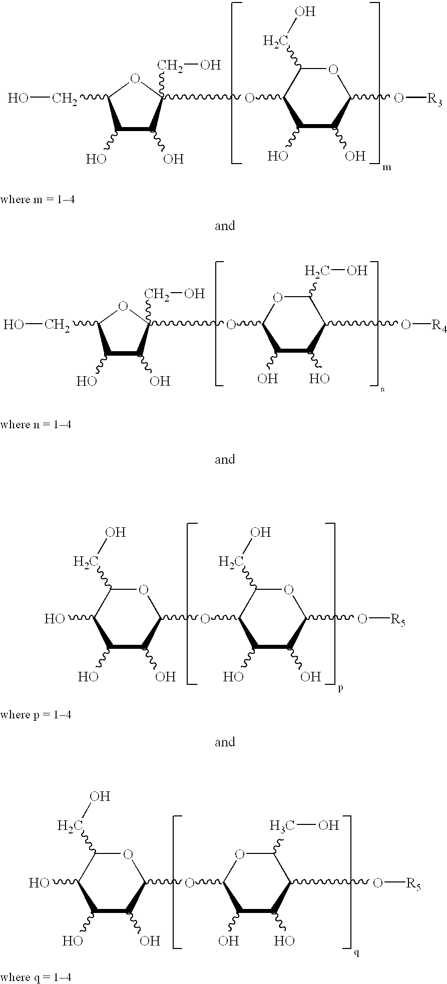
in which R₃-R₆ include branched or unbranched saturated alkyl groups or acyl groups having 1-25 carbon atoms.

4. Use according to claim 1, characterized in that the alkylated and/or acylated monosaccharides and/or oligosaccharides are present in cosmetic or dermatological formulations.

5. Use according to claim 1, characterized in that the alkylated and/or acylated monosaccharides and/or oligosaccharides are chosen from the group consisting of β-D-octylglucopyranoside, β-D-nonylglucopyranoside, β-D-decylglucopyranoside, β-D-undecylglucopyranoside, β-D-dodecylglucopyranoside, β-D-tetradecylglucopyranoside and β-D-hexadecylglucopyranoside.

6. Use according to claim 1, characterized in that the alkylated and/or acylated monosaccharides and/or oligosaccharides are present in natural or synthetic raw materials or auxiliaries or mixtures.

7. Use according to claim 4, characterized in that the alkylated and/or acylated monosaccharides and/or oligosaccharides are used in cosmetic or dermatological formulations in a content of 0.005-50.0% by weight, in particular 0.01-20.0% by weight, based on the total weight of the composition.
